(19) United States
(12) Patent Application Publication
(10) Pub. No.: US 2026/0306173 A1
(43) Pub. Date: Oct. 8, 2026

(54) LASER SKIN TREATMENT OPTICAL SYSTEM

(71) Applicant: BellaMia Technologies, Inc., San Antonio, TX (US)

(72) Inventors: Donald G. Herzog, Collingswood, NJ (US); Edward J. Adamkiewicz, Longwood, FL (US); Nick Schnauder, San Antonio, TX (US)

(73) Assignee: BellaMia Technologies, Inc., San Antonio, TX (US)

(21) Appl. No.: 19/473,982

(22) PCT Filed: Apr. 12, 2024
(86) PCT No.: PCT/US24/24241

§ 371 Date: Oct. 9, 2025

Related U.S. Application Data
(60) Provisional application No. 63/458,734, filed on Apr. 12, 2023.

(51) Int. Cl. A61B 18/20; A61B 18/00; A61B 18/22

(52) U.S. Cl. CPC A61B 18/203; A61B 18/22; A61B 2018/0047; A61B 2018/20359; A61B 2018/2222

(57) ABSTRACT

A system for providing laser energy, comprising a housing, a collimator lens disposed within the housing and configured to collimate an energy-containing beam from a source, a two-axis scanner disposed adjacent to the collimator lens and configured to controllably direct a collimated beam in 2 axes and a focal lens disposed adjacent to the two-axis scanner and configured to receive the collimated beam from the two-axis scanner and to focus the two-axis beam on a target, such as the skin of a patient.

### RELATED APPLICATIONS

The present application claims benefit of and priority to U.S. Provisional Patent Application 63/458,734, filed on Apr. 12, 2023, which is hereby incorporated by reference for all purposes as if set forth herein in its entirety.

### TECHNICAL FIELD

The present disclosure relates generally to laser energy systems, and more specifically to a laser skin treatment optical system that provides multi-wavelength control incorporated in a single system.

### BACKGROUND OF THE INVENTION

Laser treatment devices are typically dedicated to single wavelength skin treatment procedures, because of weight and size limitations, particularly with hand pieces.

### SUMMARY OF THE INVENTION

A system for providing laser energy for skin rejuvenation procedures for humans is disclosed that includes a stand-alone console and an umbilical cable from the console to a hand piece. The hand piece has a housing with an umbilical cable that couples a fiber optical input from the console to a collimator lens disposed within the housing. The housing is configured to collimate energy-containing laser beams from a fiber optic input source to a folding mirror that is used to redirect the laser beams onto a two-axis scanner. The scanner is disposed adjacent to a scan lens and is configured to controllably direct a collimated beam in two axes onto a common focal plane. The scan lens is configured to receive the collimated beam from the two-axis scanner and to focus the two-axis beam on a target. Different skin rejuvenation techniques require different laser wavelengths that can use a different and unique system to provide for a separate wavelength.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE SEVERAL VIEWS OF THE DRAWINGS

Aspects of the disclosure can be better understood with reference to the following drawings. The components in the drawings may be to scale, but emphasis is placed upon clearly illustrating the principles of the present disclosure. Moreover, in the drawings, like reference numerals designate corresponding parts throughout the several views, and in which:

FIG. 1 is a diagram of a system for laser skin rejuvenation treatment, in accordance with an example embodiment of the present disclosure;

FIG. 2 is a diagram of a system for combining different laser wavelengths into a single beam, in accordance with an example embodiment of the present disclosure;

FIG. 3 is a schematic view of a system for providing a physically small and low cost alternative approach for laser wavelength selection and combination that can select one laser at a time, in accordance with an example embodiment of the present disclosure;

FIG. 4 is a diagram of a system for a laser treatment hand piece with innovative optical components, in accordance with an example embodiment of the present disclosure;

FIG. 5 is a drawing showing an alternative embodiment that extends the focal working distance by a significant amount;

FIG. 6 is a diagram of a system for a laser treatment hand piece with overfilling, in accordance with an example embodiment of the present disclosure; and

FIG. 7 is a diagram of a system 7 for a laser treatment hand piece aperture, in accordance with an example embodiment of the present disclosure.

### DETAILED DESCRIPTION OF THE INVENTION

In the description that follows, like parts are marked throughout the specification and drawings with the same reference numerals. The drawing figures may be to scale and certain components can be shown in generalized or schematic form and identified by commercial designations in the interest of clarity and conciseness.

The present disclosure provides a number of important technical features for a laser skin treatment device, including single mirror two-dimensional (2D) scanners, the use of multiple wavelengths, unique fiber optics for wide range of wavelengths and smaller spot sizes, minimization of internal movement of optics for wavelength and spot size changes, smaller spot size, high heat dissipation aperture and a mechanically very simple rotation device to change optics.

A single mirror, two orthogonal axis scanner with a telecentric scanner output into a scan lens can provide a significant and unique approach for producing two-axis orthogonal scanning for a skin rejuvenation laser applications. The use of a physically small device but a large single mirror in a specialized mechanical mount allows it to tilt in orthogonal directions simultaneously.

The present technology in use of the skin rejuvenation industry today has significant limitations. For example, a first motor that is coupled to a first rotor that is attached to the mirror and which can rotate the mirror about a first axis, and a second motor that is coupled to a second rotor that is attached to a second mirror that can rotate the mirror about a second axis, is presently used for beam control. A housing for the mirror can include a first base on which the first motor is mounted and a second base that is coupled to the first base through the rotor attachments to the mirror, such that the second base rotates as a function of the first motor. This approach is called a dual galvanometer, which includes a motor that can rotate an attached mirror and which can be used to provide a motive force for scanning of lasers, but there are a number of problems associated with this dual galvanometer approach. There is often a tradeoff with 1) size of the mirrors to achieve increased laser beam width, 2) the scanning speed limited by the inertia, 3) the amount of power density the mirror(s) can handle, 4) the maximum scanning angle and other factors.

For a galvanometer, the inertia resulting from the mass of the motor and the mass of the mirror limits the rotational rate of the scanning process. When the mirror is larger, there is usually a higher mass to rotate, which slows the scanner. When a second galvanometer is added to a system to provide orthogonal scanning capability, another difficulty results with the mirror sizes. The first mirror provides scanning that is directed into the second mirror, which needs to be much larger to accommodate the scanning angle from the first mirror, resulting in higher inertia and thus a lower scanning rate. An additional issue with this type of orthogonal scanning is that the projected scan has what is called pincushion effect, which is a squeezing in on the center sides, which causes distortion by the second mirror because the system is not telecentric (scanning from a single location).

The use of a single mirror 2D scanner allows the system to operate with a much larger collimated beam diameter since it does not have to handle the scanning width from the first scanning mirror. The present disclosure also provides for the use of a truly telecentric optical system with very low distortion, resulting in a much more uniform skin rejuvenation procedural result.

Alternatively, a micromechanical (MEMS) scanner that uses a nanoscale mirror array can provide 2D orthogonal scanning. However, MEMS scanners have very limited effective mirror size and scan angle, and a very low laser energy handling capability. Because of these limits, MEMS scanners are not practical for skin rejuvenation, which requires a high energy handling capability, large scan angles and large mirrors to achieve small laser spot sizes onto the skin.

By using a single mirror that uses 4 independent angle drives to provide 2D orthogonal scanning, an image-space telecentric optical system is provided that avoids these and other problems with prior art solutions. The image-space telecentric optical system can be reduced to a singlet specialized double aspheric, which in combination with the 2D mirror produces a small, simplistic, and compact design while improving optical performance.

The present disclosure produces a very wide scan angle that is required for superior skin rejuvenation procedural performance with speed and high performance. The use of fewer optical components provides for higher optical energy transmission and thereby lower loss of laser energy. This feature is very important, because the laser energy is critical to successful skin rejuvenation procedures. This feature also reduces the amount of heat produced within the hand piece, and requires the movement of only one lens to change the focal distance while handling multiple wavelengths with the same optical system.

By reducing the telecentric optical system to a singlet lens, the effective focal length of the system can be kept constant while the total thickness of the optical design is reduced. This configuration effectively increases the working distance available without effecting optical performance. It also reduces the lens absorption loss and provides for a hand piece that is smaller and easier to manipulate while also handling multiple wavelengths over a wide spectral range.

The single mirror approach with its telecentric optical system provides for overall smaller optics to be used to meet improved performance over the prior art. Another advantage is an overall smaller hand piece, which allows the operator to have an improved view of the skin area where the procedure is taking place while providing for improved skin rejuvenation capability.

The present disclosure also provides optics for multiple wavelengths without the need for wavelength corrections. Existing skin rejuvenation systems typically have limited range of wavelengths within a single system or require significant component or subsystem changes to achieve a different wavelength or for a different skin rejuvenation procedure. The present disclosure requires no change of any components or subsystem to achieve multiple wavelength skin rejuvenation procedures using only one fiber optic coupling from the console to the hand piece, and no changes to the hand piece or console.

The present disclosure provides a number of important technical features. One important technical feature of the present disclosure is the use of multiple wavelength sources that can be turned on individually or simultaneously coupled into a single fiber optic conductor. Another important technical feature is a fiber optical material and design that can couple all of the provided laser energy wavelengths to the hand piece with low losses. The disclosed fiber optic configuration has a low numerical aperture, to provide for very small laser spots onto the skin. Another important technical feature is an optical system that is small, lightweight, compact and easily manipulated that can handle multiple wavelengths without any component or subsystem change. The present disclosure also helps to optimize the size and weight and to make the umbilical cable that couples the console to the hand piece less cumbersome.

The present disclosure allows each laser to be turned on, by separately triggering the laser driver to the lasers. Multiple lasers can also be simultaneously tumed on to provide combined skin rejuvenation procedures by triggering selected lasers to emit during the same period. A unique laser combiner is disclosed that utilizes dichroic mirrors that transmit one or more wavelengths and reflect one wavelength, which allow each wavelength to be superimposed over each other such that with very simple optics all wavelengths can be directed into a single fiber that will couple all the selected wavelengths to the hand piece without any mechanical motion.

Selected laser wavelengths can be directed into the hand piece and onto the skin, where a separate dichroic mirror can be used for each wavelength. For example, if only two wavelengths are incorporated into the system then only one dichroic mirror is required. The number of dichroic mirrors can thus be one less than the number of wavelengths required to be separately selectable, or other suitable configurations can also or alternatively be used. This technique can also be incorporated into the hand piece if multiple optical fibers are to be used between console and the hand piece to communicate each wavelength.

The present disclosure also provides for fiber optics having a wide range of wavelengths and smaller spot sizes. A common wavelength for skin rejuvenation is in the spectral region of 3000 nm, which requires prior art systems to use a multiple mirror system called an articulated arm that has fixed points of flexure. At each flex location of the coupling cable, a mirror is located and adjusted such that as the coupling arm is flexed, the output laser beam does not move in location. This approach is expensive and requires more frequent maintenance but is used because fiber optics to support the 3000 nm wavelengths region with low loss and high power handling have not been available.

The present disclosure uses a telluride based fiber optic system to accommodate wavelengths from 1000 nm to 4000 nm, such as a telluride glass fiber like the Mid-Infrared Transport Fiber manufactured by NP Photonics of Tucson, AZ. Telluride glass fiber eliminates the drawbacks of the typical glass fiber optics and eliminates the need for an articulated arm. A single optical fiber cable connecting the hand piece to the console can be used with the present disclosure.

A small laser spot size on the skin is preferable for many types of skin rejuvenation procedures. One way to achieve smaller laser spot sizes and also to be able to scan the spots over the skin is to use larger optics. Since it is equally important to keep the size of the hand piece to a minimum to allow it to be easily handled, this approach for smaller spots is not acceptable. The optics are a relay that includes the input spot size or numerical aperture (NA). Having the NA as small as possible reduces the output laser spot size onto the skin for the same optical system. The disclosed telluride glass fiber optic system enables the NA to be reduced, because the fiber cladding and transition from the core to the cladding can reduce the NA from typically 0.22 to 0.11, cutting the laser spot size in half. This innovation is accomplished using multiple steps of index change. One unique part of this application of the telluride glass fiber optic system is the material and the multiple step index of cladding to develop a specific NA.

The present disclosure also provides for minimization of the internal movement of optics for wavelength and spot size changes. Typically, a change in wavelength of a complex set of lenses requires a mechanical movement to maintain the same focal distance. Covering the range of wavelengths in the infrared (IR) frequency range from 1000 nm to 3000 nm is a 300% shift in wavelengths, requiring significant mechanical movement of the internal optics to maintain constant focus distance. The present disclosure provides a system lens design within the hand piece that uses compensating lens materials to handle the longitudinal chromatic shift from multiple wavelengths by using achromatized optics, such as by using achromatized collimating optics to replace a collimator lens. The use of collimating optics significantly reduces the mechanical motion of optical components to maintain the focus onto the skin.

Optomechanical components are disclosed that can handle multiple wavelengths, such as skin rejuvenation procedure wavelengths of 1470 nm, 1927 nm and 2940 nm. A single fiber can be used to couple the lasers from the console to the band piece using a telluride glass optical fiber that can handle the broad frequency spectrum, high energy, and which can support the small laser spot sizes yet be extremely rugged. The optical components can use infrared (IR) fused silica or other suitable materials that can take advantage of a high transmission across the IR to MIR spectrum and that provide attractive thermal and durability properties compared to glass or other common materials. The corrections required to maintain fiber coupling efficiency due to longitudinal chromatic shift can be performed using a precision optomechanical assembly that is much larger, or by using achromatized optics, whereby the system size is minimized.

The present disclosure provides for a smaller spot size, such as 100 um to 150 um laser spot diameters, which can be used in most circumstances to provide for a better skin rejuvenation procedure performance with faster healing. In contrast, existing systems found in the skin rejuvenation industry use dual galvanometers and often cannot reduce the laser spot size below 300 um. As discussed above, the spot size can be decreased by modifying the NA input to the hand piece, in the hand piece or in other suitable manners. One way to achieve a smaller NA is to use a pin hole type aperture in front of the fiber optic output, but this approach has certain problems, as discussed below.

Another way to achieve a smaller spot size is to move the collimator lens closer to the folding mirror, thereby overfilling the folding mirror. Since the inter beams that are reflected by the folding mirror are more highly collimated, this approach effectively lowers the NA, whereby the optics following the folding mirror will focus the laser to a smaller spot onto the skin thereby achieving a 150 um laser spot size. A significant amount of laser energy could be lost due to dissipation in that configuration, but how deep the laser energy penetrates into the skin is a function of the laser energy per square centimeter. Since the energy density is not changed when reducing the spot size from 300 um to 150 um, the penetration depth does not change.

Since the area that needs to dissipate the heat is larger, the heat dissipation can be achieved more easily. Although the folding mirror was used as an example for an over filled aperture, a separate aperture could also or alternatively be placed at a suitable location within the collimated beam, or other suitable alternatives could also or alternatively be used.

With high energy lasers and small diameter fiber optics input to the hand piece optics, pin holes or apertures have to dissipate a lot of heat in a very small volume of material, and they are also subject to reflected energy back into the laser source that can be damaging to components of the laser source. Very small pin holes or apertures are particularly subject to these conditions and are usually made from a thin structure. One alternative to a thin structure for a pin hole type aperture approach is to use an aperture where the laser beam is much larger, such as in the collimated beam region, with an aperture that is the approximate size of the input beam, and where the output is at the final aperture required. This configuration provides for a larger structure that can be configured to handle a higher heat dissipation, and which is not as likely to be damaged or to cause damage.

Adjustments for changing the focal plane distance can be made with a threaded, rotatable lens that can be manually or electronically adjusted. To correct for this large range change in focus distance, such as with a collimating lens change to a relay lens that is mechanically coupled so that by a single mechanical motion each lens is automatically replaced by the other. This two lens design approach accommodates different wavelengths and a wide range of spot sizes while keeping the overall size and weight consistent with a compact hand held device.

FIG. 1 is a diagram of a system 100 for laser skin rejuvenation treatment, in accordance with an example embodiment of the present disclosure. All major, large power consuming components or subsystems are incorporated in a stand-alone console with the objective to keep the hand piece that an operator manipulates to provide a skin rejuvenation procedure small, for easy handling.

In accordance with the skin rejuvenation systems and methods disclosed herein, it is important to make the hand piece as small and easy to manipulate as possible while providing for small laser spots at high energy over a large scan area. The uniqueness of this optical system includes the component designs and also the example embodiments that allow an integrated system of optics and lasers working together to provide for a very small laser spot size onto the skin while also providing for multiple focal distances with multiple wavelengths and a large scanning area. Different procedures require different laser wavelengths, mostly in the near to mid Infrared (IR) wavelengths. In general, these requirements are conflicting, and as one improves, the other gets worse. The present disclosure provides optimal configurations for these system components.

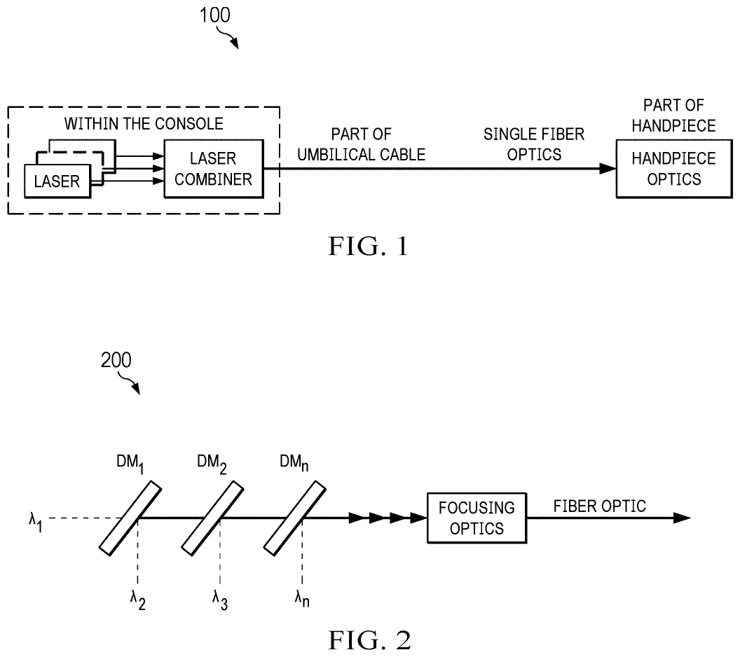

FIG. 2 is a diagram of a system 200 for combining different laser wavelengths into a single beam, in accordance with an example embodiment of the present disclosure. System 200 includes dichroic mirrors DM₁, DM₂ and DM₃, each of which are configured to pass multiple wavelengths such as λ₁ and other suitable wavelengths, and to reflect specific wavelengths λ₂, λ₃ and λ₄. In this manner, a suitable number of laser wavelengths can be combined into a single beam, where the number of combined beams is equal to or greater than the number of dichroic mirrors. Dichroic mirrors DM₁, DM₂ and DM₃ can be formed from multiple ultra-thin layers of transparent oxides of such metals as titanium, chromium, aluminum, zirconium, or magnesium, which can be vaporized by an electron beam in a vacuum chamber and then condensed on the surface of a glass or other transmittance material substrate. Physical vapor deposition (PVD) coatings or other suitable processes can also or alternatively be used. The finished glass can have a suitable number of layers of materials, with a total coating thickness having a suitable range, such as 750 to 900 nm.

System 200 allows for all laser wavelengths to be combined. Prior art skin rejuvenation systems use one laser per stand-alone system. The present disclosure combines all wavelengths in the near to mid IR wavelengths (800 nm to 4000 nm) into a single optical fiber that connects the laser energy to the hand piece and provides for all laser emitters to be located in the console. By combining the wavelengths in the console, the present disclosure minimizes the complexity and size of the hand piece. System 200 uses dichroic mirrors to provide collimated, concentric laser beams that can be directed into a single fiber optic.

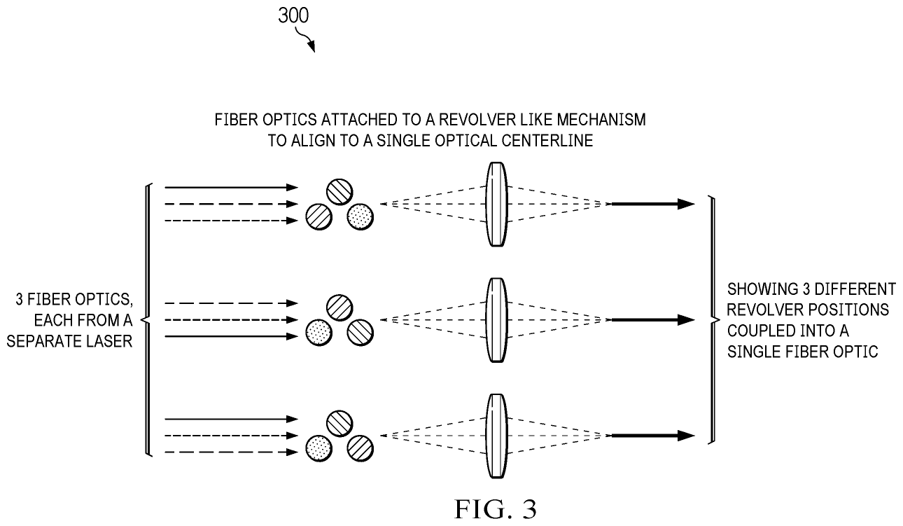

FIG. 3 is a schematic view of a system 300 for providing a physically small and low cost alternative approach for laser wavelength selection and combination that can select one laser at a time, in accordance with an example embodiment of the present disclosure. Since optical fibers are very flexible, the present disclosure can utilize a mechanism to bring all fibers (using three as an example) as close together as possible with a mechanical combiner, such as a gun-like revolver approach to rotate a barrel that centers each fiber optic output into a common fiber optic, a linear mechanical slider or other suitable configurations. This approach can use an optical lens to refocus the selected optical fiber output into a single fiber that can communicate the selected laser energy via the umbilical cable to the hand piece, where the fiber optic material for each particular laser wavelength will be plugged into the revolver. Because of its small size, the mechanical combiner could also be located in the hand piece with separate fibers connected between the console and the hand piece. An articulated set of mirrors is also provided, in combination with a telluride fiber optic material that allows the 2940 nm laser wavelength to be transmitted, which eliminates the need for an articulated coupling from the console to the hand piece.

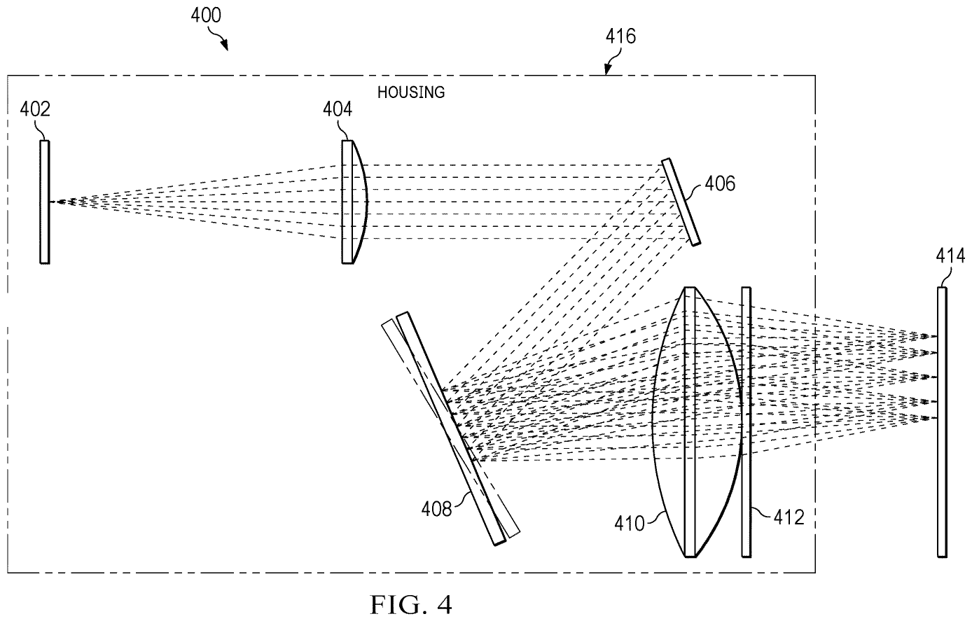

FIG. 4 is a diagram of a system 400 for a laser treatment hand piece with innovative optical components, in accordance with an example embodiment of the present disclosure. System 400 includes input 402, collimator lens 404, mirror 406, two-axis scanner 408, scan lens 410 and window 412, which can be contained within a hand piece housing 416 or other suitable housings, and focal plane 414.

Input 402 is configured to provide laser energy or other suitable forms of energy for treatment of a patient or other suitable applications. In one example embodiment, input 502 can be a pinhole or other suitable apertures to allow laser light to enter or leave an optical fiber or other suitable optical fiber light loading or supplying systems or components, including but not limited to multiplexers, combiners and adders.

Collimator lens 404 can be formed from IR fused silica or other suitable materials and can process light for transmission through an optical fiber, air, a vacuum or other suitable media. Collimator lens 404 can be configured to adjust a beam size, area or other suitable beam components.

Mirror 406 can be formed from metal, glass coated with metal or other suitable materials. In one example embodiment, mirror 406 can be a foldable mirror, an adjustable mirror or other suitable mirrors.

Two-axis scanner 408 can be a suitable two-axis scanner for adjusting a laser light beam direction or other suitable purposes. In one example embodiment, two-axis scanner 408 can be a single mirror two-dimensional scanner or other suitable devices.

Scan lens 410 can be formed from optical glass or other suitable materials that can provide good transmittance for the wavelength to be handled and can process light to separate laser beams having different frequencies. In one example embodiment, scan lens 510 can be configured to operate with MIR laser light or other suitable laser light.

Window 412 can be formed from optical glass or other suitable materials, and can be configured to protect the optical components of system 400 from outgassing, debris or other materials generated during treatment of a patient or other applications. In one example embodiment, window 412 can be replaceable, to allow it to be removed for cleaning or other suitable purposes.

Focal plane 414 can be used to calibrate system 400 for use with patients or other suitable purposes. In one example embodiment, focal plane 414 can include one or more sensors that can generate data for assessing the configuration of system 400, to determine whether system components need to be replaced or repaired or for other suitable purposes.

Hand piece housing 416 can be configured to be held in a single hand by a user and to direct laser energy to a suitable location. In one example embodiment, hand piece housing 516 can be made from plastic or other suitable materials, can include one or more ports for circulation of cooling air or for other inputs and outputs.

In operation, system 500 allows laser energy to be received from a fiber optic conductor or other suitable sources and to be effectively processed in a small, handheld device. System 500 prevents excessive heat generation and improves system operation, and uses multiple laser wavelengths sources that can be installed within the console with the ability to bring each laser wavelength over a broad range of IR wavelengths through a single optical fiber or any number of optical fibers to the hand piece.

In order to avoid using separate systems for each laser wavelength, as is common in the prior art, the present disclosure provides a system that implements a number of functional attributes:
1) the system can handle the wide range of wavelengths that are commonly provided by separate systems within the IR wavelength region;
2) the system can provide for scanning a linear array at least 20 mm wide of small laser spots onto the skin, where the laser spots are separated by approximately 1 mm; or other convenient separation; and
3) the system also provides for an orthogonal array of spots at least 15 mm 2 and separated by approximately 1 mm or other convenient separation.

The prior art uses separate hand pieces that plug into an umbilical cable end, or has totally separate consoles to achieve these requirements. The input to the disclosed system can be a wideband fiber optic conductor made of telluride material that can carry wavelengths within the near infrared (NIR) to mid infrared (MIR) frequency range. In one example embodiment, the fiber optics diameter could be 400 um and could correspond to a numerical aperture (NA) of 0.2 that would have an expanding laser beam out of the input fiber optic. This unique optical system provides for a hand piece that can effectivity use a number of input wavelengths, can be very small and easily manipulated, can handle the required laser power and energy per pulse, and can provide for a large range of spot sizes including very small laser spot sizes onto the skin in, such as 150 um spot diameter. A collimating lens can be used to collimate the laser beam, to minimize distortion created by the scanner and to provide other benefits discussed herein. A folding mirror can be used to project the laser beam onto the scanner and to help to minimize the size of the hand piece, and can also improve the overall optical performance by minimizing the size of the scanning mirror relative to a two mirror system used in the prior art, to achieve a two axis scanning.

The prior art uses a dual axis galvanometer scanner that includes two orthogonal mirrors. Since the second mirror must handle the scan distribution from the first mirror, this approach limits the size of the mirrors that can be used to support required scan rate, which also limits how small the laser spot size that is projected onto the skin can be. Alternatively, if the mirrors are made large enough to support the laser spot size, then the scan rate is reduced, which increases the skin rejuvenation procedure time. This approach is also quite large because of the size of the motors used.

A dual orthogonal axis, two mirror scanner is used in the prior art. To overcome the problems of the prior art, a single 2D scanning mirror that can scan in orthogonal axes simultaneously at very high speeds is disclosed. This type of scanner can be a Model MR-15-30 available from Optotune of Dietikon, Switzerland or other suitable scanners, and represents about one-fifth of the size of a typical galvanometer system. The output of the scanning mirror is projected into a scan lens that transfers the scanned input into orthogonal laser spots onto the skin with a selectable range of spot sizes. It is noted that mirrors made using MEMS technology lack the scan angle and power handling capability required for this application.

The scanning lens design, coupled with a single mirror 2D scanner is unique in that it produces a field-flattened optical performance with astigmatic and spherical aberration corrections. While this function could also be accomplished by an optical group of three to five spherical optics that are larger in length and diameter to produce the same performance, the present disclosure can be made significantly smaller and more compact by reducing the design to only two specialized aspheric surfaces.

The present disclosure provides for lower attenuation losses than other configurations due to reduced total substrate thickness and has less surfaces required to coat with antireflection coatings. As a result, the present disclosure is more streamlined for a batch production scheme with an assembly that only requires one component, is ultimately lower in cost, and also provides a very important greater effective working distance between the scan lens and the focal plane called focal distance.

As a result of these unique features, the present disclosure provides a higher optical performance with reduced spot size. The present disclosure can be used to provide a plurality of wavelengths, a better working distance for the operator, and a higher scan rate for shorter total skin rejuvenation procedure times, which are better for the providers and their patients. The present disclosure balances aberration corrections for two different focal distances that are used for two common types of procedures.

Another unique aspect of the scan lens design is to provide for a second and important increase to multiply the focal distance between the scan lens and the focal plane by only modifying the simple collimating lens with another simple lens. The design of the scan lens provides for field curvature aberration optimization in both the shorter focal and the longer focal distances.

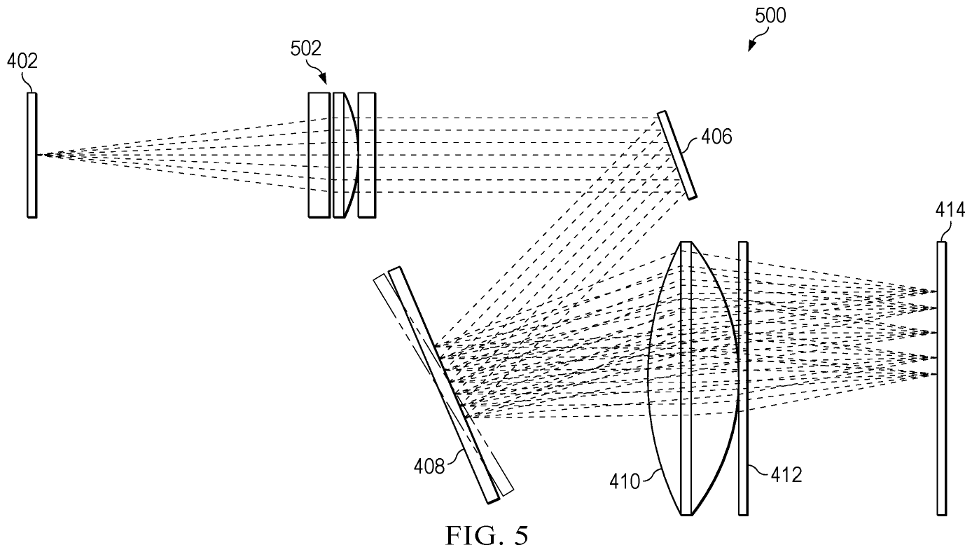

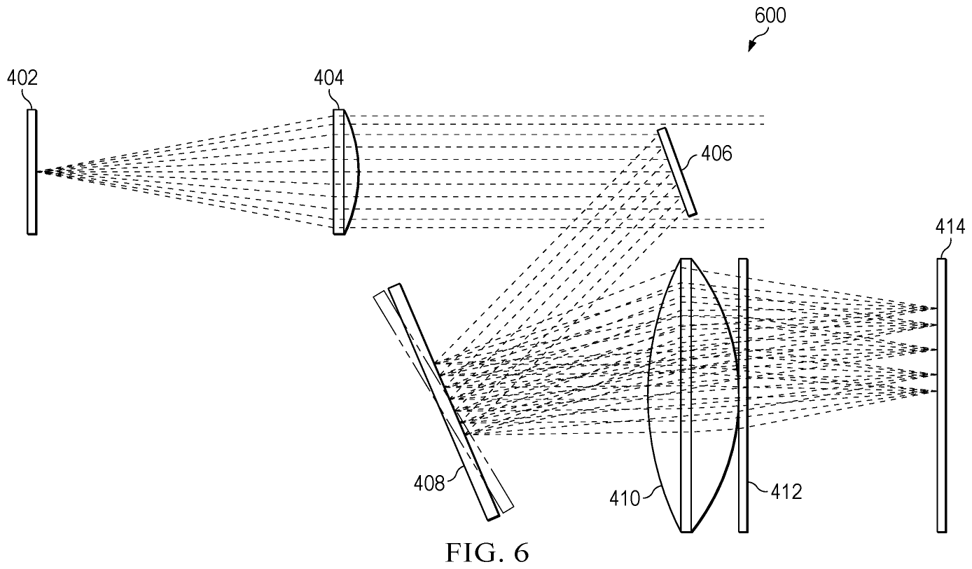

FIG. 5 is a drawing showing an alternative embodiment of system 400, that extends the focal working distance by a significant amount. The scan lens design provides for the use of an additional achromatized cylindrical optic 502 to be included in collimating lens 404 to compensate for astigmatism that usually becomes dominant in the shorter working distance. Another unique aspect of the lens design of this alternative embodiment is that the achromatized cylindrical optic 502 keeps the magnification of the deposited field of laser spots uniform for all selected wavelengths.

Another unique aspect of this system design is the use of a laser diode pumped Erbium doped YAG rod, as opposed to a flash lamp pumped light source. Flash lamps are inefficient and limit output to about 30 pulses per second. In contrast, laser diodes are about five times more efficient than flash lamps and therefore can have a pulse rate up to 300 pulse per second. One example of a suitable laser diode pumped Erbium doped YAG rod is the model number DPM-50 [Er:YAG] available from Pantec Biosolutions AG, of Tamil Nadu, India. With the higher Erbium doped YAG laser pulse rate and the higher speed single two axis mirror, the skin rejuvenation system can perform procedures much faster, making it better for the patient and the operator.

FIG. 6 is a diagram of a system 600 for a laser treatment hand piece with overfilling, in accordance with an example embodiment of the present disclosure. System 600 uses a folding mirror to allow overfilling to be utilized to change a size of a spot of laser energy, as discussed and described further herein.

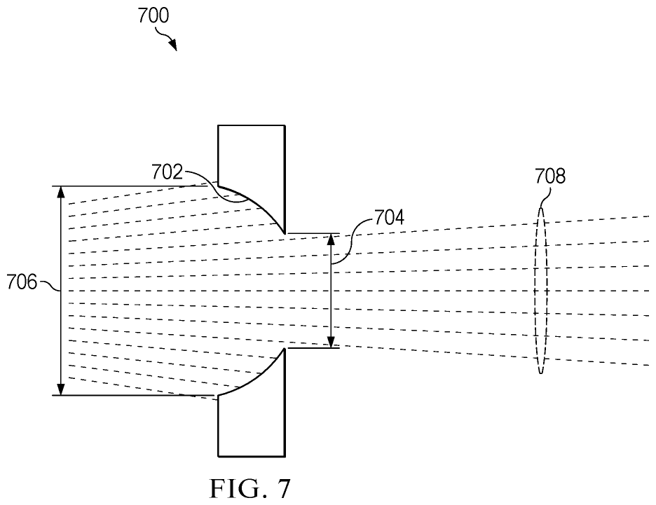

FIG. 7 is a diagram of a system 700 for a laser treatment hand piece aperture, in accordance with an example embodiment of the present disclosure. System 700 includes aperture 702, which can be formed from a material with a high thermal inertia, a material with internal cooling channels or other suitable materials. Aperture 702 includes inner diameter 704 and outer diameter 706, which can be configured to provide better beam focusing and other suitable optical features. In one example embodiment, a fixed or variable radius of curvature as shown in system 700 is used to provide improved focusing and other optical parameters. In this manner, light energy 708 can be transmitted without losing beam focus parameters necessary for treatment.

As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.” As used herein, “hardware” can include a combination of discrete components, an integrated circuit, an application-specific integrated circuit, a field programmable gate array, or other suitable hardware. As used herein, “software” can include one or more objects, agents, threads, lines of code, subroutines, separate software applications, two or more lines of code or other suitable software structures operating in two or more software applications, on one or more processors (where a processor includes one or more microcomputers or other suitable data processing units, memory devices, input-output devices, displays, data input devices such as a keyboard or a mouse, peripherals such as printers and speakers, associated drivers, control cards, power sources, network devices, docking station devices, or other suitable devices operating under control of software systems in conjunction with the processor or other devices), or other suitable software structures. In one exemplary embodiment, software can include one or more lines of code or other suitable software structures operating in a general purpose software application, such as an operating system, and one or more lines of code or other suitable software structures operating in a specific purpose software application. As used herein, the term “couple” and its cognate terms, such as “couples” and “coupled,” can include a physical connection (such as a copper conductor), a virtual connection (such as through randomly assigned memory locations of a data memory device), a logical connection (such as through logical gates of a semiconducting device), other suitable connections, or a suitable combination of such connections. The term “data” can refer to a suitable structure for using, conveying or storing data, such as a data field, a data buffer, a data message having the data value and sender/receiver address data, a control message having the data value and one or more operators that cause the receiving system or component to perform a function using the data, or other suitable hardware or software components for the electronic processing of data.

In general, a software system is a system that operates on a processor to perform predetermined functions in response to predetermined data fields. A software system is typically created as an algorithmic source code by a human programmer, and the source code algorithm is then compiled into a machine language algorithm with the source code algorithm functions, and linked to the specific input/output devices, dynamic link libraries and other specific hardware and software components of a processor, which converts the processor from a general purpose processor into a specific purpose processor. This well-known process for implementing an algorithm using a processor should require no explanation for one of even rudimentary skill in the art. For example, a system can be defined by the function it performs and the data fields that it performs the function on. As used herein, a NAME system, where NAME is typically the name of the general function that is performed by the system, refers to a software system that is configured to operate on a processor and to perform the disclosed function on the disclosed data fields. A system can receive one or more data inputs, such as data fields, user-entered data, control data in response to a user prompt or other suitable data, and can determine an action to take based on an algorithm, such as to proceed to a next algorithmic step if data is received, to repeat a prompt if data is not received, to perform a mathematical operation on two data fields, to sort or display data fields or to perform other suitable well-known algorithmic functions. Unless a specific algorithm is disclosed, then any suitable algorithm that would be known to one of skill in the art for performing the function using the associated data fields is contemplated as falling within the scope of the disclosure. For example, a message system that generates a message that includes a sender address field, a recipient address field and a message field would encompass software operating on a processor that can obtain the sender address field, recipient address field and message field from a suitable system or device of the processor, such as a buffer device or buffer system, can assemble the sender address field, recipient address field and message field into a suitable electronic message format (such as an electronic mail message, a TCP/IP message or any other suitable message format that has a sender address field, a recipient address field and message field), and can transmit the electronic message using electronic messaging systems and devices of the processor over a communications medium, such as a network. One of ordinary skill in the art would be able to provide the specific coding for a specific application based on the foregoing disclosure, which is intended to set forth exemplary embodiments of the present disclosure, and not to provide a tutorial for someone having less than ordinary skill in the art, such as someone who is unfamiliar with programming or processors in a suitable programming language. A specific algorithm for performing a function can be provided in a flow chart form or in other suitable formats, where the data fields and associated functions can be set forth in an exemplary order of operations, where the order can be rearranged as suitable and is not intended to be limiting unless explicitly stated to be limiting.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

What is claimed is:

1. A system for providing laser energy, comprising:
a housing;
a collimator lens disposed within the housing and configured to collimate an energy-containing beam from a source;
a two-axis single mirror scanner disposed adjacent to the collimator lens and configured to controllably direct a collimated beam in two axes; and
a focal lens disposed adjacent to the two-axis single mirror scanner and configured to receive the collimated beam from the two-axis single mirror scanner and to focus the two-axis beam on a target (skin).

2. The system of claim 1 wherein the two-axis scanner comprises a single mirror scanner.

3. The system of claim 1 wherein the two-axis scanner comprises a single large high performance mirror coupled to a first induction dual coil drive and by a second orthogonal induction dual coil drive.

4. The system of claim 1 wherein the two-axis scanner comprises a larger mirror with higher speed in a single two axis mirror scanner that can shorten the skin rejuvenation procedure time for patients and operators.

5. The system of claim 1 wherein the two-axis scanner comprises a larger mirror with high energy handling capability in a single mirror.

6. The system of claim 1 wherein the two-axis single mirror scanner comprises a larger mirror able to achieve smaller laser spot sizes capability in a single mirror.

7. The system of claim 1 wherein the two-axis single mirror scanner comprises a much smaller, more compact scanner able to achieve a more compact, more easily manipulated handpiece.

8. The system of claim 1 further comprising a telluride glass fiber optic system coupled to the housing and configured to deliver laser energy to a collimator lens.

9. The system of claim 1 further comprising a single telluride glass fiber optic system coupled to the housing and configured to deliver laser energy to a collimator lens using multiple wavelengths.

10. The system of claim 1 further comprising a single telluride glass fiber optic system coupled to the housing and configured to deliver laser energy at a Numerical Aperture (NA) of 0.11.

11. The system of claim 1 further comprising a single telluride glass fiber optic system coupled to the housing and configured to deliver laser energy with an optical attenuation of less than 0.5 dB per meter.

12. The system of claim 1 comprising an Erbium doped YAG laser rod, that is pumped using laser diodes and not lamp pumped.

13. The system of claim 1 comprising the ability to incorporate multiple laser wavelengths into a single system.

14. The system of claim 1 comprising the ability to incorporate one or more of the laser wavelengths to provide for one or more skin rejuvenation procedures simultaneously.

15. The system of claim 1 whereby a competitive sized hand piece can provide 2 or more operating laser wavelengths.

16. The system of claim 1 whereby an optical design, located within the hand piece, can be used with multiple wavelengths with a single fiber optic input.

17. The system of claim 1 whereby an optical design can be used with multiple wavelengths covering the near IR (Infrared) spectrum of lasers

18. The system of claim 1 whereby an optical design can be used with multiple wavelengths covering the mid IR (Infrared) spectrum of lasers.

19. A system of claim 1 whereby because of all lasers being direct output laser diodes or laser diode pumped laser rods can provide for shorter skin rejuvenation procedures because of higher laser pulse rates.

20. A system of claim 1 whereby time spent with the patient is shortened because no component, subsystem or system is required to be changed when changing procedures.
